(19) United States
(12) Patent Application Publication
(10) Pub. No.: US 2020/0060809 A1
(43) Pub. Date: Feb. 27, 2020

(54) SYSTEMS AND METHODS FOR VIBRATION DETECTION AND COMMUNICATION

(71) Applicant: Johnson & Johnson Vision Care, Inc., Jacksonville, FL (US)

(72) Inventors: Adam Toner, Jacksonville, FL (US); Randall B. Pugh, Jacksonville, FL (US)

(21) Appl. No.: 16/110,890

(22) Filed: Aug. 23, 2018

(51) Int. Cl. A61F 2/16; G02C 7/04; A61B 5/00

(52) U.S. Cl. CPC A61F 2/1624; G02C 7/04; A61B 5/103; A61B 2560/0219; A61B 5/6821

(57) ABSTRACT

The present disclosure relates to sensor systems for electronic ophthalmic devices. In certain embodiments, the sensor systems may comprise a sensor and a processor operably connected to the sensor. The processor may be configured for receiving, from the sensor, sensor data representative of a vibration caused by the user. The vibration may be caused at least in part by one or more of a mouth of the user or an extremity of the user. The processor may be configured for determining a user instruction based on the vibration. The processor may be configured for causing the ophthalmic device to be controlled based on the user instruction.

### BACKGROUND OF THE DISCLOSURE

### 1. Field of the Disclosure

The present disclosure relates to electronic ophthalmic devices, such as wearable lenses, including contact lenses, implantable lenses, including intraocular lenses (IOLs) and any other type of device comprising optical components, and more particularly, to sensors and associated hardware and software for detecting vibration and other movements made by a user to activate and control electronic ophthalmic devices.

### 2. Discussion of the Related Art

Ophthalmic devices, such as contact lenses and intraocular lenses, currently are utilized to correct vision defects such as myopia (nearsightedness), hyperopia (farsightedness), presbyopia and astigmatism. However, properly designed lenses incorporating additional components may be utilized to enhance vision as well as to correct vision defects.

Ophthalmic devices may incorporate a lens assembly having an electronically adjustable focus to augment or enhance performance of the eye. The use of embedded electronics in a lens assembly introduces a potential requirement for communication with the electronics, for a method of powering and/or re-energizing the electronics, for interconnecting the electronics, for internal and external sensing and/or monitoring, and for control of the electronics and the overall function of the lens.

Conventional contact lenses are polymeric structures with specific shapes to correct various vision problems as briefly set forth above. To achieve enhanced functionality, various circuits and components have to be integrated into these polymeric structures. For example, control circuits, microprocessors, communication devices, power supplies, sensors, actuators, light-emitting diodes, and miniature antennas may be integrated into contact lenses via custom-built optoelectronic components to not only correct vision, but to enhance vision as well as provide additional functionality.

For example, electronic and/or powered contract lenses may be designed to provide enhanced vision via zoom-in and zoom-out capabilities, or simply modify the refractive capabilities of the lenses. Electronic and/or powered contact lenses may be designed to enhance color and resolution, to display textural information, to translate speech into captions in real time, to offer visual cues from a navigation system, and to provide image processing and internet access. The lenses may be designed to allow the wearer to see in low-light conditions. The properly designed electronics and/or arrangement of electronics on lenses may allow for projecting an image onto the retina, for example, without a variable-focus optic lens, provide novelty image displays and even provide wakeup alerts. Alternately, or in addition to any of these functions or similar functions, the contact lenses may incorporate components for the noninvasive monitoring of the wearer's biomarkers and health indicators.

In addition, because of the complexity of the functionality associated with a powered lens and the high level of interaction between all of its components, there is a need to coordinate and control the overall operation of the electronics and optics.

### SUMMARY OF THE DISCLOSURE

The present disclosure relates to powered or electronic ophthalmic devices that comprise an electronic system that, in turn, performs any number of functions, including actuating a variable-focus optic if included. The electronic system may include one or more batteries or other power sources, power management circuitry, one or more sensors, clock generation circuitry, control algorithms, circuitry comprising a (e.g., displacement) sensor, and lens driver circuitry.

The present disclosure relates to an ophthalmic system comprising: an ophthalmic device configured to be disposed in or on an eye of a user; and a sensor system disposed in or on the ophthalmic device, the sensor system comprising a sensor and a processor operably connected to the sensor and configured for: receiving, from the sensor, sensor data representative of a vibration caused by the user, wherein the vibration is caused at least in part by one or more of a mouth of the user or an extremity of the user; determining a user instruction based on the vibration; and causing the ophthalmic device to be controlled based on the user instruction.

The present disclosure relates to a method comprising: receiving, from a sensor of an ophthalmic device disposed in or on an eye of a user, a vibration signal indicative of a vibration caused at least in part by a change in a characteristic of a mouth of the user; determining, based at least on the vibration signal, a user instruction; and causing the ophthalmic device to be controlled based on the user instruction.

The present disclosure relates to an ophthalmic system comprising: an ophthalmic device configured to be disposed in or on an eye of a user; a sensor configured to be disposed at least partially in a mouth of the user, wherein the sensor is configured to output sensor data representative of a movement of the user; and a processor disposed in or on the ophthalmic device and configured for: receiving the sensor data from the sensor; determining a user instruction based on the sensor data; and causing the ophthalmic device to be controlled based on the user instruction.

The present disclosure relates to a method comprising: receiving, by an ophthalmic device disposed in or on an eye of a user from a sensor configured to be disposed at least partially in a mouth of the user, sensor data; determining a user instruction based on the sensor data; and causing the ophthalmic device to be controlled based on the user instruction.

### BRIEF DESCRIPTION OF THE DRAWINGS

The foregoing and other features and advantages of the disclosure will be apparent from the following, more particular description of preferred embodiments of the disclosure, as illustrated in the accompanying drawings.

FIG. 1 illustrates an exemplary ophthalmic device comprising a sensor system in accordance with some embodiments of the present disclosure.

FIG. 2 illustrates an exemplary ophthalmic device comprising a sensor system in accordance with some embodiments of the present disclosure.

FIG. 3 is a graphical representation demonstrating correlations between measurable electrical parameters and the eye's desired focal length in accordance with the present disclosure.

FIG. 4 is a planar view of an ophthalmic device comprising electronic components, including a sensor system and a variable-optic element in accordance with the present disclosure.

FIG. 5A is a diagrammatic representation of an exemplary electronic system incorporated into an ophthalmic device in accordance with the present disclosure.

FIG. 5B is an enlarged view of the exemplary electronic system of FIG. 5A

FIG. 6 illustrates a schematic diagram of an exemplary integrator in accordance with some embodiments of the present disclosure.

FIG. 7 illustrates a schematic diagram of an exemplary integrator in accordance with some embodiments of the present disclosure.

FIG. 8 illustrates a schematic diagram of an exemplary out-of-bounds circuit in accordance with some embodiments of the present disclosure.

FIG. 9 is a diagrammatic representation of an exemplary powered or electronic ophthalmic device in accordance with the present disclosure.

FIG. 10 is a flowchart illustrating an example method in accordance with the present disclosure.

FIG. 11 is a flowchart illustrating another example method in accordance with the present disclosure.

### DETAILED DESCRIPTION OF THE PREFERRED EMBODIMENTS

Ophthalmic devices may include implantable device and/or wearable devices, such as contact lenses. Conventional contact lenses are polymeric structures with specific shapes to correct various vision problems as briefly set forth above. To achieve enhanced functionality, various circuits and components may be integrated into these polymeric structures. For example, control circuits, microprocessors, communication devices, power supplies, sensors, actuators, light-emitting diodes, and miniature antennas may be integrated into contact lenses via custom-built optoelectronic components to not only correct vision, but to enhance vision as well as provide additional functionality as is explained herein.

Electronic and/or powered ophthalmic devices such as contact lenses may be designed to provide enhanced vision via zoom-in and zoom-out capabilities, and/or to modify the refractive capabilities of the lenses. Electronic and/or powered devices may be designed to enhance color and resolution, to display textural information, to translate speech into captions in real time, to offer visual cues from a navigation system, and to provide image processing and internet access. The devices may be designed to allow the user/wearer to see in low light conditions. The properly designed electronics and/or arrangement of electronics on devices (e.g., lenses) may allow for projecting an image onto the retina, for example, without a variable focus optic lens, provide novelty image displays and even provide wakeup alerts. Alternately, or in addition to any of these functions or similar functions, the ophthalmic devices (e.g., contact lenses) may incorporate components for the noninvasive monitoring of the wearer's biomarkers and health indicators. For example, sensors built into the ophthalmic devices (e.g., contact lenses) may allow a diabetic patient to keep tabs on blood sugar levels by analyzing components of the tear film without the need for drawing blood. In addition, an appropriately configured lens may incorporate sensors for monitoring cholesterol, sodium, and potassium levels, as well as other biological markers. This coupled with a wireless data transmitter could allow a physician to have almost immediate access to a patient's blood chemistry without the need for the patient to waste time getting to a laboratory and having blood drawn. In addition, sensors built into the ophthalmic devices (e.g., contact lenses) may be utilized to detect light incident on the eye to compensate for ambient light conditions or for use in determining blink patterns.

The powered or electronic ophthalmic devices of the present disclosure may comprise the necessary elements to correct and/or enhance the vision of patients with one or more of the above described vision defects or otherwise perform a useful ophthalmic function. In addition, the electronic contact lens may be utilized simply to enhance normal vision or provide a wide variety of functionality as described above. The electronic contact lens may comprise a variable focus optic lens, an assembled front optic embedded into a contact lens or just simply embedding electronics without a lens for any suitable functionality. The electronic lens of the present disclosure may be incorporated into any number of contact lenses as described above. In addition, intraocular lenses may also incorporate the various components and functionality described herein. However, for ease of explanation, the disclosure will focus on an electronic contact lens to correct vision defects intended for single-use daily disposability.

The present disclosure may be employed in a powered ophthalmic device comprising an electronic system, which actuates a variable-focus optic or any other device or devices configured to implement any number of numerous functions that may be performed. The electronic system includes one or more batteries or other power sources, power management circuitry, one or more sensors, clock generation circuitry, control algorithms and circuitry, and lens driver circuitry. The complexity of these components may vary depending on the required or desired functionality of the lens.

Control of an electronic or a powered ophthalmic lens may be accomplished through a manually operated external device that communicates with the lens, such as a hand-held remote unit. For example, a fob may wirelessly communicate with the powered lens based upon manual input from the wearer. Alternately, control of the powered ophthalmic lens may be accomplished via feedback or control signals directly from the wearer. For example, sensors built into the lens may sense signals indicative of ciliary muscle movement, i.e. contraction and relaxation, to compensate for crystalline lens dysfunction or any other problems associated with visual acuity or eye disease. Based upon these signals, the powered ophthalmic lens may change state, for example, its refractive power, in order to either focus on a near object or a distant object.

The ophthalmic devices may have sensors configured to detect other movement, such as gestures indicating that a user intends to communicate an instruction to the ophthalmic device. The sensor may be disposed in or on ophthalmic device. As another example, the sensor may be located remotely from the ophthalmic device, such as in a mouth, belt, or other place on the user. The sensor may be configured to detect movement and/or vibrations caused by the user, including movement of the user's mouth. The user may perform chattering, moving, grinding, or clenching a tooth of the user to communicate an instruction to the ophthalmic device. These movements may cause vibrations. The sensor may detect the vibrations as the vibrations reach the ophthalmic device. In some implementations, a sensor may be disposed in a user's mouth, such as in a cap placed over a tooth. The sensor in the cap may detect contact between the teeth and transmit sensor data to the ophthalmic device, which may analyze the sensor data to determine whether the movements indicates that the user is communicating with the ophthalmic device.

The systems, devices, and methods of the present disclosure may be configured to sense movement of the ciliary muscle of a user, for example, using vibration sensing. The ciliary muscle in the eye is the structure that controls or attempts to control the shape of the crystalline lens. The crystalline lens is encased in the capsule which is suspended by zonules connected to the ciliary muscle. The ciliary muscle causes the zonules to contract or to relax thereby changing the shape and/or focusing power of the crystalline lens. Vibration caused by the ciliary muscle may be detected along with vibrations from other movements, such as movement of the mouth. The user may combine multiple gestures including movement of the eyes and/or movements of other body parts, such as the mouth to indicate an instruction to the ophthalmic device.

Powered or electronic ophthalmic devices may have to account for the various ciliary muscle signals detected from an individual utilizing the powered or electronic ophthalmic devices. More specifically, powered ophthalmic devices may need to detect and differentiate between various ciliary muscle signals (e.g., vibrations), and from one or more of other signals, noise, and interference. Ophthalmic devices may also be configured to differentiated between vibrations caused by other parts of the body, such as movement of the mouth, that are intended as an instruction to the ophthalmic device and other common movements. For example, the ophthalmic device may distinguish between chewing and clenching of the teeth to communicate an instruction to ophthalmic device.

Accordingly, it should be understood that a sensor in accordance with the present disclosure may provide feedback signals for controlling any number of functions that may be implemented by a powered or electronic ophthalmic lens. However, in accordance with the present disclosure, the circuitry is configured to detect, isolate and amplify signals intended as communication to the ophthalmic device (e.g., vibration caused by the mouth, or eye movements) while filtering out noise and other muscle signals.

A sensor, the components of which may be embedded in a powered contact lens, or may be disposed in a user's mouth may detect characteristics of different movements of the user. For example, various signals may include one or more of when an eye is moving up or down, focusing up close, and adjusting to a change in ambient light levels, such as from light to dark, dark to light or any other light condition. The ciliary muscle controls the shape of the crystalline lens in order to focus on a near or distant object. The sensor relies on tracking various signals, including amplitude, time-domain response and frequency composition, produced by or emitted from the ciliary muscle in certain sample conditions, such as when an individual is reading, focusing far away, or in a room with fluorescent lighting. It is important to note that this list of conditions is exemplary and not exhaustive.

These movement signal samples may be logged and tracked wherein the various waveforms and frequencies of each of the signals may be distinguished from one or more of other signals, noise, and interference. As set forth above, the circuitry of the present disclosure is preferably designed to detect, isolate and/or filter ciliary muscle signals. In alternate embodiments, other muscle signals may be utilized for augmenting or implementing other ocular functions. Whenever the sensor detects a recognized movement signal, the sensor may trigger activity in the electronic circuitry, for example, activating an electronic lens. Other activity may comprise deactivating the ophthalmic lens, changing a setting, determining an acknowledgment, determining a gesture and corresponding instruction and/or command to perform based on the gesture, and/or the like.

There may be various methods used to implement some exemplary embodiments of the present disclosure. For example, sensors may detect movement signals utilizing displacement (e.g., vibration) sensing and/or a microphone, alone or in combination with, one or more of electromyography (EMG), magnetomyography (MMG), phonomyography (PMG), and impedance, and/or the like. Furthermore, sensors may comprise a non-contact sensor, such as an antenna that is embedded into a contact lens, but that does not directly touch the surface of an eye. Alternately, sensors may comprise a contact sensor, such as contact pads that directly touch the surface of an eye, oral contact sensors that directly touch the surface of a tooth (e.g., tongue or other part of mouth), and/or the like. It is important to note that any number of suitable devices and processes may be utilized for the detection of signals from the ciliary muscle as is explained in detail subsequently. As described herein, any type of sensor and/or sensing technology may be utilized.

In certain embodiments, ophthalmic devices may comprise an ophthalmic lens having an optic zone and a peripheral zone. Ophthalmic devices may comprise a variable optic element incorporated into the optic zone of the ophthalmic lens, the variable optic being configured to change the refractive power of the wearable ophthalmic lens. Ophthalmic devices may comprise a sensor, such as a vibration sensor disposed in the peripheral zone of the ophthalmic lens, the vibration sensor configured to detect a vibration. The vibration may be based on a movement of a mouth, such as movement of teeth (e.g., against each other). The vibration may be based on movement of the eye and/or ciliary muscles of the eye. The vibration may be based on other movements, such as tapping of a foot, hand, arm, leg, finger, and/or the like. A processor may determine a user instruction based on the vibration signal and cause output of a signal to control the ophthalmic device based on the user instruction.

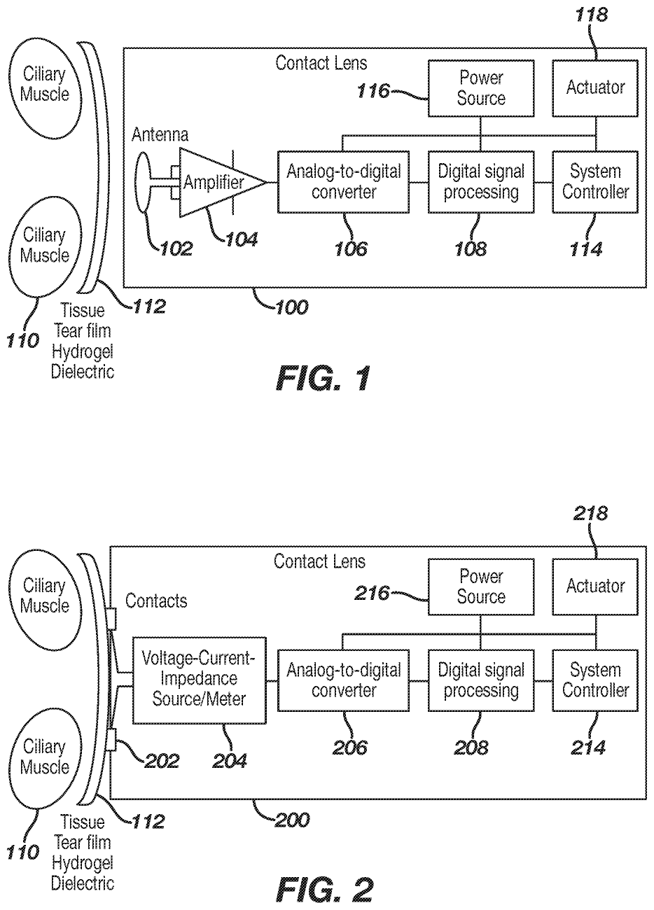

FIG. 1 illustrates, in block diagram form, an ophthalmic device 100 disposed on the front surface of the eye or cornea 112, in accordance with one exemplary embodiment of the present disclosure. Although the ophthalmic device 100 is shown and described as a being disposed on the front surface of the eye, it is understood that other configurations, such as those including intraocular lens configuration may be used. In this exemplary embodiment, the sensor system may comprise one or more of a sensor 102, a sensor circuit 104, an analog-to-digital converter 106, a digital signal processor 108, a power source 116, an actuator 118, and a system controller 114. As illustrated, the ciliary muscle 110 is located behind the front eye surface or cornea 112. More specifically, the globe of the eye can be divided into two segments; namely, the anterior chamber and the posterior chamber. The iris is the partition between the anterior and posterior chambers. Between the front surface of the crystalline lens and the back surface of the iris is the posterior chamber. At the base of the iris is the ciliary body which produces aqueous humor and is continuous with the ciliary muscle. The ophthalmic device 100 is placed onto the front surface of the eye 112 wherein the electronic circuitry of the sensor system may be utilized to implement the neuromuscular sensing of the present disclosure. The sensor 102 as well as the other circuitry is configured to sense signals from ciliary muscle 110 actions through the various tissue and liquids forming the eye and produced by the eye. The sensor 102 may also sense vibration signals transmitted via the body of the user from other movements. For example, if a user taps his teeth together, then a vibration signal may travel through the body to the eye and be detected by the sensor 102. As another example, if a user taps his or her foot or other limb, this may also generate a vibration signal that is transmitted through the body and detected via one or more sensors, disposed in the eye, the mouth, on a wrist, on a belt, in a shoe, and/or elsewhere. As set forth above, the various fluids comprising the eye are good conductors of electrical and acoustical signals.

In this exemplary embodiment, the sensor 102 may be at least partially embedded into the ophthalmic device 100. The sensor 102 may also be embedded in a tooth cap disposed on a tooth of a user. The sensor may be disposed elsewhere, such as on a belt, on a wrist (e.g., in a band, activity tracker, or a watch), in a shoe, and/or the like. The sensor 102 may be in mechanical communication with the eye, for example disposed to sense vibration associated with (e.g., translating through) the eye. The sensor 102 may be in mechanical communication with the mouth (e.g., tooth) or other extremity (e.g., leg, arm, hand) of the user. The sensor 102 may be or comprise one or more components configured to sense a displacement (e.g., vibration) at or near the eye, the mouth, the arm, the hand, the finger, the leg, and/or the like. The sensor 102 may comprise a micro ball sensor, a piezo vibration sensor, a cantilever sensor, a microphone, and the like. The sensor 102 may comprise a piezoelectric, sonic, subsonic, and/or ultrasonic sensor component. The sensor 102 may comprise an emitter/detector pair. The sensor 102 may be configured to generate an electrical signal indicative of the sensed vibration. As such, when characteristics of the ciliary muscle change, the sensor 102 may sense displacement(s) due to such change and may generate the electrical signal indicative of such change or resultant characteristic. For example, there may be various signals detected by the sensor 102 depending on the state that a ciliary muscle is in, such as whether it is contracting or relaxing, or on the type of action that a ciliary muscle is trying to perform, such as causing the eye to focus on a near object or a far object. As another example, there may be various signals detected by the sensor 102 depending on the state of the user's mouth, tooth, tongue, arm, leg, foot, hand, finger, and/or the like.

As a further example, particular states of the user's body (e.g., ciliary muscle, tooth, other extremity) representing one or more characteristics of the body at a given time, may be associated with a particular displacement signature indicative of the particular state. Additionally or alternatively, the change between states body may be associated with a particular displacement signature indicative of the particular transition between states. A set of displacement signatures may be determined (e.g., via experimentation) and may be stored for subsequent comparison. The set of displacement signatures may be generated using machine learning, heuristics, signal processing, and/or comparison to one or more predetermined signatures. The set of displacement signatures may be user specific and/or time specific based on actual or predictive use patterns over a period of time.

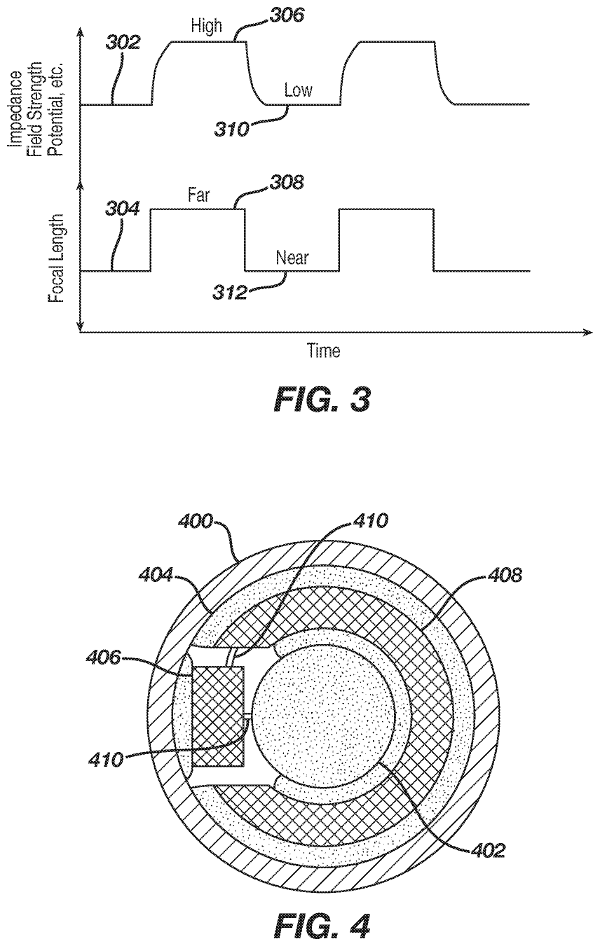

Example signatures include those associated with the ciliary muscle contracting and relaxing in response to an accommodative stimulus to change lens focus. Peak intensity of muscle movement may occur when the stimulus changes near/far or far/near, which may be represented by a derivative of the signals 302, 306 (FIG. 3). This muscle movement causes a corresponding change in tension and movement of the zonules and lens. A characteristic signal associated with such ciliary muscle movement, translated through the zonules and eye to an appropriate sensor, may have distinctly different characteristics in amplitude, duration, and frequency than other signals around the eye. For example, natural accommodation occurs over a period of hundreds of milliseconds and involves both fast changes in reaction to stimulus change and slow changes to maintain focus as part of a feedback loop. Signal processing can differentiate between the fast changes, slow changes, and other signals such as eye movements. As an example, data captured via one or more sensors and/or sensor systems of the present disclosure may be processed based on comparative data such as maximum velocities of saccades and microsaccades of relative to amplitudes, main-sequence diagrams showing peak velocity, duration, and the first peak acceleration as a function of saccadiac magnitude for the saccadic eye movement, and/or main sequence disparity vergence responses, for example. Such processing (e.g., comparison, filtering, etc.) may facilitate the differentiation of noise and may be used to differentiate between the fast changes, slow changes, and other signals such as eye movements. Other comparative data may be collected and used to process the information captured via the sensors and sensor systems of the present disclosure.

Example signatures include those associated with blinking of the eye, moving the eye left or right, moving the eye up or down, and/or the like. Example signatures include those associated with moving a mouth, such as biting, tapping, clenching, grinding, and/or the like of one or more teeth of the user. Example signatures include those associated with moving an extremity, such as any vibration due to movement of an arm, hand, finger, foot, leg, head, and/or the like. As an example, data captured via one or more sensors and/or sensor systems of the present disclosure may be processed based on comparative data such predetermined signal characteristics. The predetermined signal characteristics may be specific to the user. For example, during a calibration mode, the user may perform different movements, such as biting teeth, moving jaw, winking an eye, tapping an arm/hand against a side of the user, and/or the like. The signal characteristics may be used to determine specific movements.

The system controller 101 may analyze one or more movements during regular operation, and/or during a gesture window. The gesture window may be a time period for performing one or more gestures. The ophthalmic device may be configured to indicate that a gesture window is beginning and/or ending. For example, one or more movements (e.g., or vibration signals caused by the movements) may be stored during the gesture window. The one or more movements may be matched to corresponding movements associated with commands, functions, communications, instructions, acknowledgements, and/or the like.

Analysis of sensor data may comprise categorization (e.g., matching) of sensor data based on one or more movements. The data may be associated with and/or comprise time values (e.g., to track different vibration over time). A set of changes of vibration over time may be analyzed to determine specific gestures. Frequency, duration, amplitude values, and/or the like may be matched to a gesture and/or user communication. An example gesture may comprise user tapping of teeth of the user in a pattern, such as X number of quick taps followed by Y number of longer taps, where X and Y can be any whole numbers. An example gesture may comprise the user grinding the teeth back and forth in a pattern, such as forward and backward any number of times. The example gesture may comprise tapping a tooth on one side of the mouth followed by taping a tooth on the other side of the mouth. The sensor may determine which side of the body and/or mouth a vibration originated from based on any character. The system controller 114 may be configured to determine which part (e.g., left side, right side, mouth, tooth, lower body, upper body, arm, leg, foot, hand, finger) of the body a signal originated from. For example, the amplitude, frequency, and/or the like may be indicative of the part of the body. Accordingly, the signal may be matched to a communication, instruction, command, and/or function based on a determination of which part of the body originated the vibration.

Once gestures are determined, a corresponding command may be determined (e.g., by the system controller 114). The command may be executed to cause a change in a setting, change in a context, navigate a menu, change a mode, and/or perform any other operation for which the ophthalmic device may be configured. As explained further herein, the sensor data may also comprise blink detection data, vibration data related to the eye, capacitance data, impedance data, and/or the like. Sensor data from one or more sensors may be analyzed separately or together to determine whether one or more movements are intended as a gesture by the user.

Returning to FIG. 1, the sensor circuit 104 or sensor system may be configured to process signals received by the sensor 102. As an example, the sensor circuit 104 may be configured to amplify a signal to facilitate integration of small changes in signal level. As a further example, the sensor circuit 104 may be configured to amplify a signal to a useable level for the remainder of the system, such as giving a signal enough power to be acquired by various components of the sensor circuit 104 and/or the analog-to-digital converter 106. In addition to providing gain, the sensor circuit 104 may include other analog signal conditioning circuitry such as filtering and impedance matching circuitry appropriate to the sensor 102 and sensor circuit 104 output. The sensor circuit 104 may comprise any suitable device for amplifying and conditioning the signal output by the sensor 102. For example, the sensor circuit 104 may simply comprise a single operational amplifier or a more complicated circuit comprising one or more operational amplifiers.

As set forth above, the sensor 102 and the sensor circuit 104 are configured to capture and isolate the signals indicative of characteristic of the ciliary muscle from the noise and/or other signals produced in or by the eye and convert it to a signal usable ultimately by the system controller 114. The system controller 114 is preferably preprogrammed to recognize the various signals produced by the ciliary muscle under various conditions and provide an appropriate output signal to the actuator 118. The sensor 102 and the sensor circuit 104 may be configured to capture and isolate the signals indicative of characteristic of other user movements (e.g., vibration caused by movement of teeth, mouth, arm, leg, hand) and convert it to a signal usable ultimately by the system controller 114. The system controller 114 is preferably preprogrammed to recognize the various signals produced by the mouth, arms, legs, hands, and/or the like that are intended as communication to the ophthalmic device under various conditions and provide an appropriate output signal to the actuator 118.

In this exemplary embodiment, the analog-to-digital converter 106 may be used to convert an analog signal output from the amplifier into a digital signal for processing. For example, the analog-to-digital converter 106 may convert an analog signal output from the sensor circuit 104 into a digital signal that may be useable by subsequent or downstream circuits, such as a digital signal processing system 108 or microprocessor. A digital signal processing system or digital signal processor 108 may be utilized for digital signal processing, including one or more of filtering, processing, detecting, and otherwise manipulating/processing sampled data to discern a ciliary muscle signal from noise and interference. The digital signal processor 108 may be preprogrammed with the user movement signatures and/or patterns described above. The digital signal processor 108 may be implemented utilizing analog circuitry, digital circuitry, software and/or preferably a combination thereof. For example, various vibration signals that may occur within a certain frequency range may be distinguishable from other signals, noise, and interference that occur within other frequency ranges. Certain commonly occurring noise and interference signals may be notched at various stages in the signal acquisition chain utilizing analog or digital filters, for example, harmonics of 50/60 Hz AC mains and fluorescent lights. It may be advantageous to filter various noise and interference signals through a combination of analog and digital signal processing, for example to use differential circuit design techniques to reject common-mode noise that could overload a sensitive amplifier, while performing time- and frequency-domain analysis (e.g. to differentiate ciliary muscle signals from eye movements, or to differentiate movements or vibrations indicative of a user instruction from other user movements) in digital signal processing.

A power source 116 supplies power for numerous components comprising the non-contact sensor system. The power may be supplied from a battery, energy harvester, or other suitable means as is known to one of ordinary skill in the art. Essentially, any type of power source may be utilized to provide reliable power for all other components of the system. A vibration signal (e.g., from movement of the mouth, or ciliary muscles), processed from analog to digital, may enable activation of the system controller 114. Furthermore, the system controller 114 may control other aspects of a powered contact lens depending on input from the digital signal processor 108, for example, changing the focus or refractive power of an electronically controlled lens through an actuator 118.

In further alternate exemplary embodiments, the system controller 114 may receive input from sources including one or more of a vibration sensor, contact sensor, a blink detector, and a fob control. By way of generalization, it may be obvious to one skilled in the art that the method of activating and/or controlling the system controller 114 may require the use of one or more activation methods. For example, an electronic or powered contact lens may be programmable specific to an individual user, such as programming a lens to recognize both of an individual's vibration signals when performing various actions, for example, focusing on an object far away, focusing on an object that is near, an individual's blink patterns, movement of a mouth (e.g., grinding, biting, clenching, etc of teeth, or movement of tongue), movement of other extremity (e.g., stomping feet, tapping finger or hand against something). In some exemplary embodiments, using more than one method to activate or otherwise control an electronic contact lens, such as vibration signal detection and blink detection, may give the ability for each method to crosscheck with another before activation of the contact lens occurs. For example, vibration due to ciliary muscles may be analyzed along with vibration due to other user movements, such as moving mouth, teeth, or other extremities. An advantage of crosschecking may include mitigation of false positives, such as minimizing the chance of unintentionally triggering a lens to activate or determining any other user instruction.

In one exemplary embodiment, the crosschecking may involve a voting scheme, wherein a certain number of conditions are met prior to any action taking place. The actuator 118 may comprise any suitable device for implementing a specific action based upon a received command signal. The actuator 118 may comprise an electrical device, a mechanical device, a magnetic device or any combination thereof. The actuator 118 receives a signal from the system controller 114 in addition to power from the power source 116 and produces some action based on the signal from the system controller 114. For example, if the system controller 114 signal is indicative of the wearer trying to focus on a near object, the actuator 118 may be utilized to somehow change the refractive power of the electronic ophthalmic lens.

FIG. 2 illustrates an ophthalmic device 200, comprising a sensor system, shown on the front surface of the eye or cornea 112 in accordance with another exemplary embodiment of the present disclosure. In this exemplary embodiment, a sensor system may comprise a contact or multiple contacts 202, a sensor circuit 204, an analog-to-digital converter 206, a digital signal processor 208, a power source 216, an actuator 218, and a system controller 214. The ciliary muscle 110 is located behind the front eye surface or cornea 112. The ophthalmic device 200 is placed onto the front surface of the eye 112, such that the electronic circuitry of the sensor may be utilized to implement the neuromuscular sensing of the present disclosure. The components of this exemplary system are similar to and perform the same functions as those illustrated in FIG. 1, with the exception of contacts 202 and the sensor circuit 204. In other words, since direct contacts 202 are utilized, there is no need for an antenna or an amplifier to amplify and condition the signal received by the antenna.

In the illustrated exemplary embodiment, the contacts 202 may provide for a direct electrical connection to the tear film and the eye surface. For example, the contacts 202 may be implemented as metal contacts that are exposed on the back curve of the ophthalmic device 200 and be made of biocompatible conductive materials, such as gold or titanium. Furthermore, the contact lens polymer may be molded around the contacts 202, which may aid in comfort on the eye and provide improved conductivity through the ophthalmic device 200. Additionally, the contacts 202 may provide for a low resistance connection between the eye's surface 112 and the electronic circuitry within the ophthalmic device 200. Four-terminal sensing, also known as Kelvin sensing, may be utilized to mitigate contact resistance effects on the eye. The sensor circuit 204 may emit a signal with several constituent frequencies or a frequency sweep, while measuring the voltage/current across the contacts 202.

In an alternate exemplary embodiment, the sensor circuit 204 may be configured to sense a vibration produced by the contraction or relaxation of the ciliary muscle 110 and/or a vibration caused by other body movement (e.g., movement of teeth, mouth, limbs). It is important to note that various types of sensors may be utilized, given that the eye comprises various fluids, including tears which are excellent conductors. The sensor circuit 204 may be configured to measure vibration, wherein the vibration may change based upon what a ciliary muscle is trying to do, such as contracting or relaxing, and/or what other movements the user is performing. In this exemplary embodiment, the analog-to-digital converter 206 and the digital signal processing 208 may be configured differently for a contact-based sensor as opposed to a non-contact based sensor, as described in FIG. 1. For example, there may be a different sample rate, a different resolution, and different signal processing algorithm.

FIG. 3 illustrates a graph demonstrating correlations between measurable electrical parameters and the eye's focal length as described in the referenced literature. Trace 302 is a representation of an electrically measurable signal in or on the eye. For example, such signals may be detected as one or more of impedance, voltage potential, induced electromagnetic field, and other measurable parameters (e.g., displacement). Trace 304 is a representation of a desired focal length wherein for example, if clinical subjects focused on objects at 0.2 and 2.0 meter distances, the ciliary muscle may undergo a corresponding change in measurable electrical parameters and displacement characteristics accordingly, depending on the distance of focus. However, using the same example, the actual focal length of a lens may not change or only changes minimally, such as in cases where a person may be presbyopic and the lens of the eye is too rigid and unable to accommodate for a change in focus, even where the ciliary muscles are responding to the change.

As described in the literature, there is a correlation between a measurable electrical signal and a focal length. As illustrated in FIG. 3, impedance is high 306 when the focal length is far 308 and impedance is low 310 when the focal length is near 312. Additionally, as described in the literature but not illustrated in FIG. 3, a correlation exists between the amplitude of traces 302 and 304 for intermediate values. Moreover, displacement signatures may be associated (e.g., correlated) with a particular state of the ciliary muscle and/or transitions between such states, which may also be associated with an impedance and/or change in such impedance.

In some exemplary embodiments, characteristics of an electrical signal (e.g., trace 302, 304) such as shape, frequency content, timing, and amplitude, may vary due to several factors including one or more of a detection method utilized (e.g., vibration, impedance, or field strength), an individual's eye physiology, ciliary muscle fatigue, electrolyte levels in the eye, state of presbyopia, interference, and focal length. For example, depending on the type of detection method used, the correlation between desired focus and measurable electrical parameter may have the opposite polarity from what is illustrated in FIG. 3.

Additionally, for example, a signal may be distorted from carrying one or more of significant noise, interference from other muscles, and interference from various environmental sources or due to the effects of aging, disease or genetics. Accordingly, studies of eye response and individual user measurement and training may be used to program the digital signal circuitry to properly detect the eye's desired focal length. Parameters of the digital signal processing may be adjusted in response to other measurements, for example, time of day, measured electrolyte levels, ambient light levels and the like. Furthermore, recorded samples of a user's eye focus signals may be used in conjunction with interference detection and mitigation techniques. It is important to note that any type of sensor may be utilized in accordance with the present disclosure. As long as there is muscle movement associated with changing conditions, it may be sensed, processed and utilized to enhance, augment or simply provide vision correction.

Sensor data related to movements of the eye may be analyzed with sensor data due to other user movements, such as movements of the mouth. A sensor may be configured to detect vibrations due to ciliary muscle movements, vibrations due to tooth contact, vibrations due to other mouth movements, vibrations due to movements of the user's extremities, and/or the like. Multiple sensors may be used, such as a contact sensor disposed on a mouth of a user, a sensor disposed in the ophthalmic device and/or the like. Similar to the signals show in FIG. 3, the vibration signals from these various sensors may be matched to a known signature, such as signatures and/or patterns associated with communications from the user.

Referring now to FIG. 4, there is illustrated, in planar view, a wearable electronic ophthalmic device comprising a sensor in accordance with the present disclosure. The ophthalmic device 400 comprises an optic zone 402 and a peripheral zone 404. The optic zone 402 may function to provide one or more of vision correction, vision enhancement, other vision-related functionality, mechanical support, or even a void to permit clear vision. In accordance with the present disclosure, the optic zone 402 may comprise a variable optic element configured to provide enhanced vision at near and distant ranges based on signals sensed from the ciliary muscle. The variable-optic element may comprise any suitable device for changing the focal length of the lens or the refractive power of the lens based upon activation signals from the sensing system described herein. For example, the variable optic element may be as simple as a piece of optical grade plastic incorporated into the lens with the ability to have its spherical curvature changed. The peripheral zone 404 comprises one or more of electrical circuits 406, a power source 408, electrical interconnects 410, mechanical support, as well as other functional elements.

The electrical circuits 406 may comprise one or more integrated circuit die, printed electronic circuits, electrical interconnects, and/or any other suitable devices, including the sensing circuitry described herein. The power source 408 may comprise one or more of battery, energy harvesting, and or any other suitable energy storage or generation devices. It is readily apparent to the skilled artisan that FIG. 4 only represents one exemplary embodiment of an electronic ophthalmic lens and other geometrical arrangements beyond those illustrated may be utilized to optimize area, volume, functionality, runtime, shelf life as well as other design parameters. It is important to note that with any type of variable optic, the fail-safe is distance vision. For example, if power were to be lost or if the electronics fail, the wearer is left with an optic that allows for distance vision.

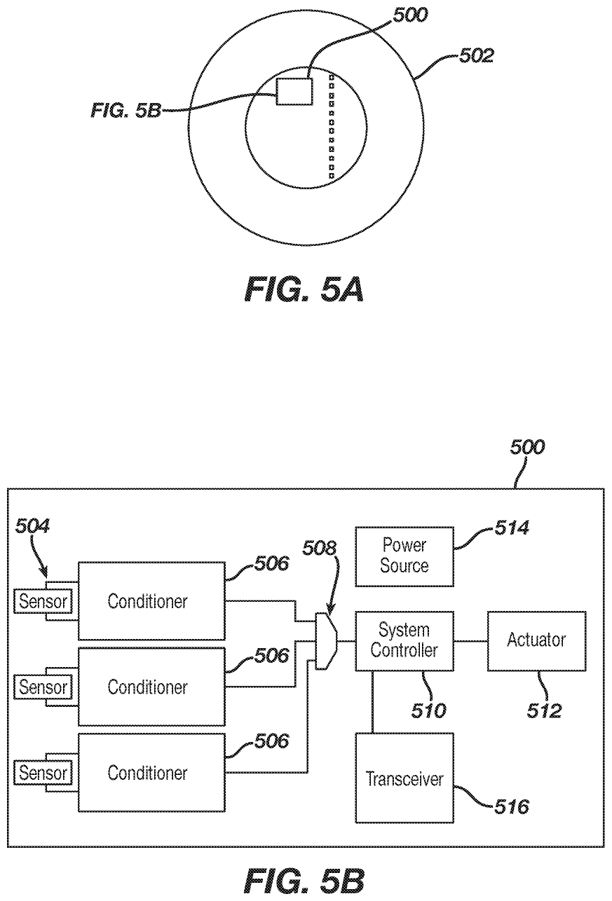

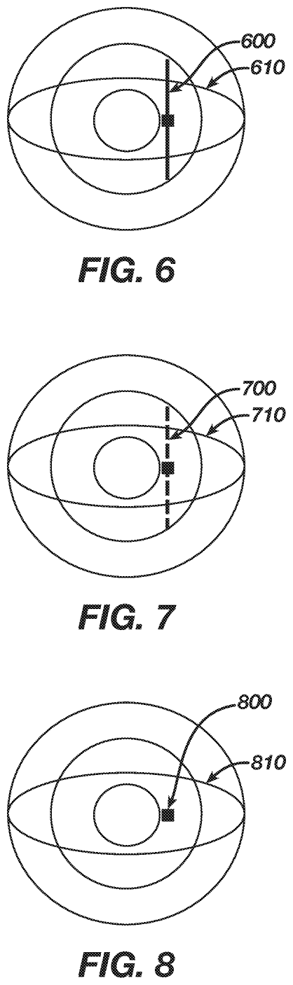

FIGS. 5A and 5B illustrate an alternate exemplary detection system 500 incorporated into an ophthalmic device 502 such as a contact lens. FIG. 5A shows the system 500 on the ophthalmic device 502 and FIG. 5B shows an exemplary schematic view of the system 500. In this exemplary embodiment, vibration sensors 504 may be used to sense a displacement at and/or adjacent an eye of the user of the ophthalmic device 502. As an example, one or more of the vibration sensors 504 may be configured to detect a displacement that may be affected by a configuration of the ciliary muscle of the user. One or more of the vibration sensors 504 may be configured to detect vibrations from other user movements, such as movements in the mouth (e.g., tapping, biting, grinding, clenching teeth) and movements of extremities (e.g., tapping foot, hand, finger or arm). One or more of the vibration sensors 504 may be configured as linear sensor 600 (FIG. 6), a segmented sensor 700 (FIG. 7), and/or an integrating sensor 800 (FIG. 8) configured to integrate a response over a sensor area. In the various configurations illustrated in FIGS. 6-8, the sensors 600, 700, 800 may be configured to sense a vibration due at least in part to a configuration of the ciliary muscle. As explained herein, one or more sensors may also be disposed in a mouth of a user, such as a contact sensor disposed in or on a tooth cap.

Returning to FIGS. 5A and 5B, sensor conditioners 506 create an output signal indicative of a measurement of one or more sensors 504 in communication with a respective one or more of the sensor conditioners 506. For example, the sensor conditioners may amplify and or filter a signal received from a respective sensor 504. The output of the sensor conditioners 506 may be combined with a multiplexer 508 to reduce downstream circuitry.

In certain embodiments, downstream circuitry may include a system controller 510, which may comprise an analog-to-digital converter (ADC) that may be used to convert a continuous, analog signal into a sampled, digital signal appropriate for further signal processing. For example, the ADC may convert an analog signal into a digital signal that may be useable by subsequent or downstream circuits, such as a digital signal processing system or microprocessor, which may be part of the system controller 510 circuit. A digital signal processing system or digital signal processor may be utilized for digital signal processing, including one or more of filtering, processing, detecting, and otherwise manipulating/processing sampled data. The digital signal processor may be preprogrammed with various displacement signatures. As an example, a data store of vibration (e.g., displacement) measurements or signatures may be mapped to particular configurations of the ciliary muscle, other conditions relating to the eye, movements of the user's mouth, movements of the users extremities, and/or the like. As such, when vibration measurements matching or near a particular signature are detected, the associated ciliary muscle characteristic, user instruction, and/or configuration may be extrapolated. Although reference is made to the ciliary muscle configuration, other conditions relating to the eye may be extrapolated such as gaze and/or accommodation. The digital signal processor also comprises associated memory. The digital signal processor may be implemented utilizing analog circuitry, digital circuitry, software, and/or preferably a combination thereof.

The system controller 510 receives inputs from the sensor conditioner 506 via a multiplexer 508, for example, by activating each sensor 504 in order and recording the values. It may then compare measured values to pre-programmed patterns and historical samples to determine a movement signature (e.g., vibration signature due to tapping teeth or other limbs), pattern, characteristic, and/or the like. It may then activate a function in an actuator 512, for example, causing a variable-focus lens to change to a closer focal distance. In some implementations other functions may be activated, such as communication via a transceiver. The vibration sensors 504 may be laid out in a physical pattern similar to that previously described and shown in references to FIGS. 1-2 and 6-9, but would be optimized for detecting characteristics and/or changes in configurations of the ciliary muscle and other vibrations caused by user movements (e.g., movements of mouth, teeth, limbs). The sensors 504, and/or the whole electronic system, may be encapsulated and insulated from the saline contact lens environment. Various configurations of the sensors 504 may facilitate optimal sensing conditions as the ophthalmic device 502 shifts or rotates.

A power source 514 supplies power for numerous components comprising the lid position sensor system 500. The power source 514 may also be utilized to supply power to other devices on the contact lens. The power may be supplied from a battery, energy harvester, or other suitable means as is known to one of ordinary skill in the art. Essentially, any type of power source 514 may be utilized to provide reliable power for all other components of the system. A vibration sensor array pattern, processed from analog to digital, may enable activation of the system controller 510 or a portion of the system controller 510. Furthermore, the system controller 510 may control other aspects of a powered contact lens depending on input from the multiplexer 508, for example, changing the focus or refractive power of an electronically controlled lens through the actuator 512.

In one exemplary embodiment, the electronics and electronic interconnections are made in the peripheral zone of a contact lens rather than in the optic zone. In accordance with an alternate exemplary embodiment, it is important to note that the positioning of the electronics need not be limited to the peripheral zone of the contact lens. All of the electronic components described herein may be fabricated utilizing thin film technology and/or transparent materials. If these technologies are utilized, the electronic components may be placed in any suitable location as long as they are compatible with the optics. The activities of the digital signal processing block and system controller (system controller 510 in FIG. 5B) depend on the available sensor inputs, the environment, and user reactions. The inputs, reactions, and decision thresholds may be determined from one or more of ophthalmic research, pre-programming, training, and adaptive/learning algorithms. For example, the general characteristics of ciliary muscle configuration may be well-documented in literature as well as vibration characteristics due to other body movements (e.g., teeth, mouth, jaw, arm), applicable to a broad population of users, and pre-programmed into system controller. However, an individual's deviations from the general expected response may be recorded in a training session or part of an adaptive/learning algorithm which continues to refine the response in operation of the electronic ophthalmic device. In one exemplary embodiment, the user may train the device by activating a handheld fob or user device, such as a smartphone, which communicates with the device, when the user desires near focus. A learning algorithm in the device may then reference sensor inputs in memory before and after the fob signal to refine internal decision algorithms. This training period could last for one day, after which the device would operate autonomously with only sensor inputs and not require the fob.

In an aspect, the system 500 (e.g., ophthalmic device and/or sensor system) may comprise a transceiver 516. The transceiver 516 may be configured to send and receive communication signals. The transceiver 516 may be configured to communicate with remote device, such as a mobile device, an access point (e.g., cell tower), a sensor, and/or the like. The transceiver 516 may be configured to transmit using any modulation technique, such as amplitude modulation, multiplexing, and/or the like. The transceiver 516 may transmit and/or receive radio wave signals, infrared signals, ultrasonic signals, and/or the like. The radio wave signals and/or ultrasonic signals may be transmitted through a portion of the body, for example, from a sensor (e.g., disposed in a mouth of the user) to the system 500. For example, the ultrasonic signals may be transmitted through one or more bones, tissues, fluids, and/or the like in the head (e.g., or skull) of the user. The ultrasonic signals may be transmitted as vibrations to the transceiver 516 of the system 500. The ultrasonic signals may be generated by created pressure waves in the ultrasonic frequency range (e.g., over 20,000 Hz).

As an illustration of ultrasound transmission, the transceiver 516 may comprise an ultrasound module. A corresponding ultrasound module (e.g., comprising the same features) may be disposed in a sensor (e.g., disposed in the user's mouth, disposed in tooth cap). The illustrated ultrasound module may comprise a digital signal processor, an oscillator, a burst generator, a transmit driver, a transmit ultrasound transducer, an analog signal processor, a receive amplifier, a receive ultrasound transducer, a combination thereof, and/or the like. In at least one embodiment, the burst generator produces a series of l's and 0's to facilitate communication with another lens and/or an external device, such as a sensor (e.g., sensor disposed in the mouth of the user). In at least one alternative embodiment for the ultrasound module, the digital signal processor is combined with the system controller 510.

The digital signal processor receives a control signal from the system controller 510. In at least one embodiment, the digital signal processor includes a resettable counter and a time-to-digital convertor and transmit/receive sequencing controls. The oscillator in at least one embodiment is a switched oscillator. The frequency of the oscillator is programmable through a preset value, the system controller or external interface. The frequency can be tuned using a reference oscillator and an external interface. In at least one embodiment, the frequency is set or tuned to a value that minimizes transmit and receive electrical power and allows the transmit ultrasound transducer to produce a pressure sound wave that will have maximum amplitude at the receiver input. In a more particular embodiment, the oscillator is a programmable frequency oscillator such as a current starved ring oscillator where the current and the capacitance control the oscillation frequency where the frequency can be altered by changing the current supplied to the oscillator. In at least one embodiment, the wavelength of the sound pressure wave is tuned based on the dimensions of the transducer used. In a further embodiment, the oscillator varies over time for optimal transmission characteristics. In a still further embodiment, the frequency is calibrated using a reference frequency provided through an external interface and an automatic frequency control (AFC) circuit. The frequency is preset with the AFC tuning it. The frequency can be directly set through the serial interface, which is accessed through the external communications link.

In an embodiment where time of flight is used, the counter in the digital signal processor begins to count pulses outputted from the oscillator. The burst generator gates the oscillator signal for a fixed amount of time defined as the burst length. In at least one embodiment, the burst length is programmable or determined by static timing relationships within the burst generator 113.

The output voltage of the burst generator may be level shifted to the appropriate value for the transmit driver and the transmit ultrasound transducer. An example of the transmit ultrasound transducer is a piezo electric device which converts applied burst voltage to a sound pressure burst. In a further embodiment, the transmit ultrasound transducer is made of any piezo electric material that is compatible with the power source and the physical properties of the contact lens. The sound pressure wave produced by the transmit ultrasound transducer propagates from the contact lens into the field of view, from a sensor through a portion body of the user to the ophthalmic device, from the ophthalmic device through a portion of the body of the user to the sensor, and/or the like. The speed of sound in air typically is 343 meters/second, so in an embodiment that measures time of flight, then the distance to the object can be measured by dividing the travel time between the propagation of the sound pressure wave and receipt of the reflected sound pressure wave by the receive ultrasound transducer.

The receive amplifier and the analog signal processor in at least one embodiment are turned on with the oscillator or turned on after a predetermined delay after the oscillator is started. In an embodiment where the receive amplifier and the analog signal processor are started with the oscillator, the receive amplifier will receive an output from the receive ultrasound transducer proximate to when the sound pressure wave is output by the transmit ultrasound transducer. This output from the receive ultrasound transducer can be used to reset the counter in the digital signal processor. In a further embodiment, the detection of the transmitted sound pressure wave can be used as an indicator that a true transmit signal has been generated.

A sound pressure wave received by the receive ultrasound transducer will produce a voltage signal with a frequency and burst length properties related to the transmitted sound pressure wave. The voltage signal is amplified by the receive amplifier before being sent to the analog signal processor, which in an alternative embodiment to other embodiments having the receive amplifier and the signal processor are combined into a signal processor. The analog signal processor may include, but is not limited to, frequency selective filtering, envelope detection, integration, level comparison and/or analog-to-digital conversion. The analog signal processor produces a received signal that represents the received sound pressure wave at the receive ultrasound transducer, which in implementation will have a slight delay. The received signal is passed from the analog signal processor to the digital signal processor. When transmission time is used, the digital signal processor will stop the counter that is counting pulses from the oscillator when the received signal is received. In such an embodiment, the measured time can be compared to a predetermined value to determine whether a change in focus should occur. In other embodiments, the digital signal processor interprets the received signal for a message from, for example, the other contact lens or an external device. The resulting output from the digital signal processor is provided to the system controller 510.

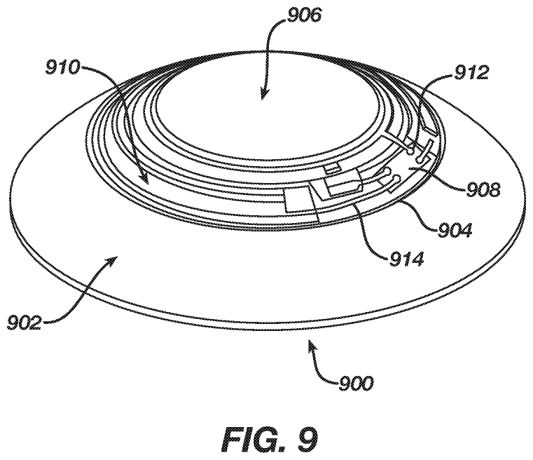

FIG. 9 is a diagrammatic representation of an exemplary electronic insert, including a combined blink detection and communication system, positioned in a powered or electronic ophthalmic device in accordance with the present disclosure. As shown, a contact lens 900 comprises a soft plastic portion 902 which comprises an electronic insert 904. This insert 904 includes a lens 906 which is activated by the electronics, for example, focusing near or far depending on activation. Integrated circuit 908 mounts onto the insert 904 and connects to batteries 910, lens 906, and other components as necessary for the system. The integrated circuit 908 includes a sensor 912 and associated signal path circuits. The sensor 912 may comprise any sensor configuration such as those described herein. The sensor 912 may also be implemented as a separate device mounted on the insert 904 and connected with wiring traces 914.

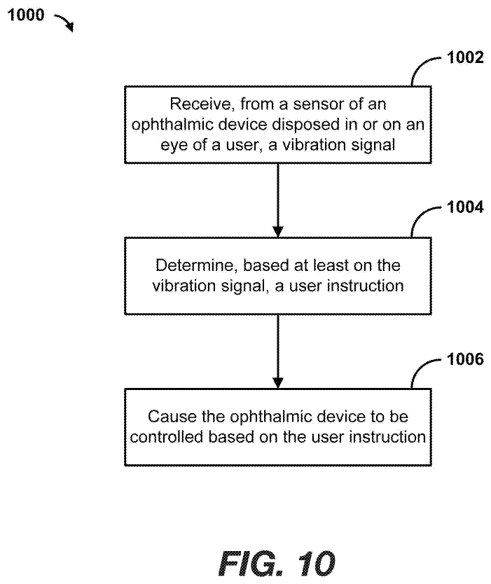

FIG. 10 is a flowchart illustrating an example method 1000 in accordance with the present disclosure. At step 1002, a vibration signal may be received. The vibration signal may be received from a sensor of an ophthalmic device disposed in or on an eye of a user. The sensor may comprise a displacement sensor, an accelerometer, a contact sensor, a combination thereof, and/or the like.

The ophthalmic device may comprise an ophthalmic lens and a variable-optic element configured to change a refractive power of the ophthalmic lens. The ophthalmic device may comprise a transceiver configured to transmit data from the ophthalmic device. The transceiver may transmit data using radio frequency waves, infrared waves, ultrasonic waves, and/or the like. The ophthalmic device may comprise an amplifier, an analog-to-digital converter, and/or the like. The method 1000 may further comprise amplifying the vibration signal. The method 1000 may further comprise converting the amplified vibration signal from an analog signal to a digital signal.

The vibration signal may be indicative of a vibration caused at least in part by a change in a characteristic of a mouth of the user. The vibration may comprise a sound made by the user. The vibration may comprise one or more of tapping, biting, chattering, moving, grinding, and/or clenching a tooth of the user. The vibration may comprise speech by the user. The vibration may comprise a tap by the extremity of the user. The extremity of the user may comprise a foot, a hand, an arm, a leg, or a finger.

At step 1004, a user instruction may be determined. The user instruction may be determined based on the vibration signal. Determining the user instruction based on the vibration may comprise determining a pattern in the vibration and determining that the pattern matches a pattern associated with the user instruction. Determining the user instruction based on the vibration may comprise determining a signature in the vibration and determining that the signature matches a signature associated with the user instruction. For example, the pattern, signature and/or the like may be associated with a gesture. Different frequencies, amplitudes, waveform shape, and/or the like may be associated with different movements. The movements may be gestures associated with corresponding user instructions. The number of times a movement is performed, intensity of the movement, and/or the like may be used to determine a corresponding user instruction.

Determining the user instruction based on the vibration signal may comprise filtering out sensor data indicative of user movements not intended as a user instruction. Filtering out sensor data indicative of user movements not intended as a user instruction may comprise filtering out sensor data in one or more of a time domain or a frequency domain.

At step 1006, the ophthalmic device may be caused to be controlled based on the user instruction. Causing the ophthalmic device to be controlled based on the user instruction may comprise causing the variable-optic element to be controlled based on the user instruction. Causing the ophthalmic device to be controlled based on the user instruction comprises causing the transceiver to be controlled based on the user instruction. As a further example, the user instruction may be executed to cause a change in a setting (e.g., calibration setting, accommodation setting, vergence setting), change in a context, navigate a menu, change a mode (e.g., driving mode, reading mode, sports mode), and/or perform any other operation for which the ophthalmic device may be configured.

As an illustration, the sensor may be disposed in or on the ophthalmic device. A user may want to indicate that he or she wants to change to a reading mode. The user may grind his or her teeth back and forth one or more times to indicate that the user wishes to enter reading mode. The sensor may detect the vibration caused by the grinding. The sensor may output a signal indicative of the grinding. The processor may match the grinding to an instruction to enter reading mode. For example, the user may use a customization feature to indicate that grinding of teeth relates to activating/deactivating reading mode. During reading mode, the ophthalmic lens may be adjusted to assist the user in reading text from a specific distance. Thus, the processor may cause the variable-optic element to adjust to a setting associated with reading mode. A similar process may be followed for adjusting to other modes, such as driving mode (e.g., accommodation setting for driving), sports mode (e.g., accommodation setting for exercise and/or sports).

As an illustration, a user may make a clicking noise. The sensor may be configured to determine frequency of the signal. The frequency may be indicative of a particular path, such as a path through the user's skull. A variety of frequencies and/or amplitudes, and/or other signal characteristics may be associated with corresponding types of signal. The frequency, amplitude, and/or other characteristics may be used to determine that the vibration signal is indicative of a clicking noise. The clicking noise may be associated with navigating a menu. For example, clicking once may cause menu items to be selected, cycled through, and/or the like. Clicking twice may cause navigation to a higher menu. The menu may be projected to the eye of the user, be an audio menu, be displayed on a screen (e.g., via an app of a mobile device), and/or the like.

As another illustration, a user may perform a teeth chattering pattern. The sensor may output vibration signal data. A second sensor, such as one detecting ciliary muscle changes or other changes in eye movement, may output additional sensor data. A third sensor may output light signal data, related to blinking of the user. The processor may determine that the teeth chattering pattern is or is not performed in combination with blinking and/or eye movement. For example, if the chattering pattern is followed by a blinking pattern, a communication message may be determined. For example, a particular text message may be associated with this combination of gestures or user movements. The ophthalmic device may send a message to a nearby mobile device to send the communication message, or the ophthalmic device may be directly connected to a wireless access point (e.g., or cell tower) and transmit the message as a text message, email, and/or the like. If the chattering pattern is followed by a different eye movement (e.g., looking left), then the processor may determine a different message to send. As another example, if the chattering pattern is not followed by another gesture, then the processor may determine that the chattering pattern is related to an acknowledgment. The acknowledgement may be part of a calibration sequence, such as calibration of an accommodation threshold, and/or the like.

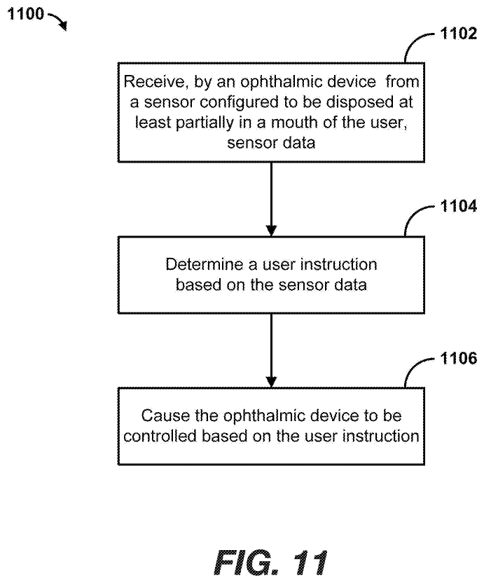

FIG. 11 is a flowchart illustrating an example method 1100 in accordance with the present disclosure. At step 1102, sensor data may be received from a sensor. The sensor data may be received, by an ophthalmic device. The ophthalmic device may be disposed in or on an eye of a user. The ophthalmic device may comprise an ophthalmic lens and a variable-optic element configured to change a refractive power of the ophthalmic lens. The ophthalmic device may comprise a transceiver configured to transmit data from the ophthalmic device. The transceiver may transmit data using radio frequency waves, infrared waves, ultrasonic waves, and/or the like. The ophthalmic device may comprise an amplifier operatively associated with the sensor. The ophthalmic device may comprise an analog-to-digital converter operatively associated with the sensor.

The sensor may be configured to be disposed at least partially in a mouth of the user. The sensor may be disposed in or on a cap configured to attach to a tooth of the user. The sensor may comprise a contact sensor. The sensor data may be indicative of a contact with one or more of a tooth or a tongue of a user. The sensor may comprise a displacement sensor, accelerometer, and/or the like.

The sensor data may be indicative of a vibration. The vibration may comprise one or more of a sound made by the user or speech by the user. The sensor data may be indicative of one or more of tapping, biting, chattering, moving, grinding, or clenching a tooth of the user. The sensor data may be indicative of movement of tongue (e.g., against the inside of the mouth, against a tooth, to make a clicking noise, etc), a jaw, a combination thereof, and/or the like.

At step 1104, a user instruction may be determined based on the sensor data. Determining the user instruction based on the sensor data may comprise determining a pattern in the sensor data and determining that the pattern matches a pattern associated with the user instruction. Determining the user instruction based on the sensor may comprise determining a signature in the sensor data and determining that the signature matches a signature associated with the user instruction. For example, the pattern, signature and/or the like may be associated with a gesture. Different frequencies, amplitudes, waveform shape, and/or the like may be associated with different movements. The movements may be gestures associated with corresponding user instructions. The number of times a movement is performed, intensity of the movement, and/or the like may be used to determine a corresponding user instruction.

Determining the user instruction based on the sensor data may comprise filtering out sensor data indicative of user movements not intended as a user instruction. Filtering out sensor data indicative of user movements not intended as a user instruction may comprise filtering out sensor data in one or more of a time domain or a frequency domain.

At step 1106, the ophthalmic device may be caused to be controlled based on the user instruction. Causing the ophthalmic device to be controlled based on the user instruction may comprise causing the variable-optic element to be controlled based on the user instruction. Causing the ophthalmic device to be controlled based on the user instruction may comprise causing the transceiver to be controlled based on the user instruction. As a further example, the user instruction may be executed to cause a change in a setting (e.g., calibration setting, accommodation setting, vergence setting), change in a context, navigate a menu, change a mode (e.g., driving mode, reading mode, sports mode), and/or perform any other operation for which the ophthalmic device may be configured.

As an illustration, a user may have the sensor disposed in the user's mouth in a tooth cap attached to the user's tooth. The ophthalmic device may be configured to adjust a setting, such as activating the lens. The user may decide that he or she does not want the lens to be activated. The user may tap one or more of this teeth together. The sensor may be a contact sensor that triggers a signal when contact is made between two teeth during the tapping motion. The sensor may transmit data indicative of the signal to a receiver in the ophthalmic device. The ophthalmic device may then analyze the signal to match the signal to a pattern. For example, the ophthalmic device may determine that the signal indicates that the user wants the lens deactivated. The ophthalmic device may then deactivate the lens.

Although shown and described in what is believed to be the most practical and preferred embodiments, it is apparent that departures from specific designs and methods described and shown will suggest themselves to those skilled in the art and may be used without departing from the spirit and scope of the disclosure. The present disclosure is not restricted to the particular constructions described and illustrated, but should be constructed to cohere with all modifications that may fall within the scope of the appended claims.

What is claimed is:

1. An ophthalmic system comprising:
a ophthalmic device configured to be disposed in or on an eye of a user; and
a sensor system disposed in or on the ophthalmic device, the sensor system comprising a sensor and a processor operably connected to the sensor and configured for:
receiving, from the sensor, sensor data representative of a vibration caused by the user, wherein the vibration is caused at least in part by one or more of a mouth of the user or an extremity of the user;
determining a user instruction based on the vibration; and
causing the ophthalmic device to be controlled based on the user instruction.

2. The ophthalmic system of claim 1, wherein the ophthalmic device comprises an ophthalmic lens and a variable-optic element configured to change a refractive power of the ophthalmic lens, and wherein causing the ophthalmic device to be controlled based on the user instruction comprises causing the variable-optic element to be controlled based on the user instruction.

3. The ophthalmic system of claim 1, wherein the ophthalmic device comprises a transceiver configured to transmit data from the ophthalmic device, and wherein causing the ophthalmic device to be controlled based on the user instruction comprises causing the transceiver to be controlled based on the user instruction.

4. The ophthalmic system of claim 1, wherein the vibration comprises a sound made by the user.

5. The ophthalmic system of claim 1, wherein the vibration comprises one or more of chattering, moving, grinding, or clenching a tooth of the user.

6. The ophthalmic system of claim 1, wherein the vibration comprises speech by the user.

7. The ophthalmic system of claim 1, wherein the vibration comprises a tap by the extremity of the user.

8. The ophthalmic system of claim 1, wherein the extremity of the user comprises a foot, a hand, an arm, a leg, or a finger.

9. The ophthalmic system of claim 1, wherein determining the user instruction based on the vibration comprises determining a pattern in the vibration and determining that the pattern matches a pattern associated with the user instruction.

10. The ophthalmic system of claim 1, wherein determining the user instruction based on the vibration comprises determining a signature in the vibration and determining that the signature matches a signature associated with the user instruction.

11. The ophthalmic device of claim 1, further comprising an amplifier operatively associated with the sensor.

12. The ophthalmic device of claim 1, further comprising an analog-to-digital converter operatively associated with the sensor.

13. The ophthalmic device of claim 1, wherein the sensor comprises a displacement sensor.

14. The ophthalmic device of claim 1, wherein determining the user instruction based on the vibration comprises filtering out sensor data indicative of user movements not intended as a user instruction.

15. A method comprising:
receiving, from a sensor of an ophthalmic device disposed in or on an eye of a user, a vibration signal indicative of a vibration caused at least in part by a change in a characteristic of a mouth of the user;
determining, based at least on the vibration signal, a user instruction; and
causing the ophthalmic device to be controlled based on the user instruction.

16. The method of claim 15, wherein the ophthalmic device comprises an ophthalmic lens and a variable-optic element configured to change a refractive power of the ophthalmic lens, and wherein causing the ophthalmic device to be controlled based on the user instruction comprises causing the variable-optic element to be controlled based on the user instruction.

17. The method of claim 15, wherein the ophthalmic device comprises a transceiver configured to transmit data from the ophthalmic device, and wherein causing the ophthalmic device to be controlled based on the user instruction comprises causing the transceiver to be controlled based on the user instruction.

18. The method of claim 15, wherein the vibration comprises a sound made by the user.

19. The method of claim 15, wherein the vibration comprises one or more of chattering, moving, grinding, or clenching a tooth of the user.

20. The method of claim 15, wherein the vibration comprises speech by the user.

21. The method of claim 15, wherein the vibration comprises a tap by the extremity of the user.

22. The method of claim 15, wherein the extremity of the user comprises a foot, a hand, an arm, a leg, or a finger.

23. The method of claim 15, wherein determining the user instruction based on the vibration comprises determining a pattern in the vibration and determining that the pattern matches a pattern associated with the user instruction.

24. The method of claim 15, wherein determining the user instruction based on the vibration comprises determining a signature in the vibration and determining that the signature matches a signature associated with the user instruction.

25. The method of claim 15, further comprising amplifying the vibration signal.

26. The method of claim 25, further comprising converting the amplified vibration signal from an analog signal to a digital signal.

27. The method of claim 15, wherein the sensor comprises a displacement sensor.

28. The method of claim 15, wherein determining the user instruction based on the vibration signal comprises filtering out sensor data indicative of user movements not intended as a user instruction.

29. The method of claim 28, wherein filtering out sensor data indicative of user movements not intended as a user instruction comprises filtering out sensor data in one or more of a time domain or a frequency domain.

30. An ophthalmic system comprising:
an ophthalmic device configured to be disposed in or on an eye of a user;
a sensor configured to be disposed at least partially in a mouth of the user, wherein the sensor is configured to output sensor data representative of a movement of the user; and
a processor disposed in or on the ophthalmic device and configured for:
receiving the sensor data from the sensor;
determining a user instruction based on the sensor data; and
causing the ophthalmic device to be controlled based on the user instruction.

31. The ophthalmic system of claim 30, wherein the sensor is disposed in or on a cap configured to attach to a tooth of the user.

32. The ophthalmic system of claim 30, wherein the sensor comprises a contact sensor.

33. The ophthalmic system of claim 30, wherein the sensor data is indicative of a contact with one or more of a tooth or a tongue of a user.

34. The ophthalmic system of claim 30, wherein the ophthalmic device comprises an ophthalmic lens and a variable-optic element configured to change a refractive power of the ophthalmic lens, and wherein causing the ophthalmic device to be controlled based on the user instruction comprises causing the variable-optic element to be controlled based on the user instruction.

35. The ophthalmic system of claim 30, wherein the ophthalmic device comprises a transceiver configured to transmit data from the ophthalmic device, and wherein causing the ophthalmic device to be controlled based on the user instruction comprises causing the transceiver to be controlled based on the user instruction.

36. The ophthalmic system of claim 30, wherein the sensor data is indicative of a vibration.

37. The ophthalmic system of claim 36, wherein the vibration comprises one or more of a sound made by the user or speech by the user.

38. The ophthalmic system of claim 30, wherein the sensor data is indicative of one or more of chattering, moving, grinding, or clenching a tooth of the user.

39. The ophthalmic system of claim 30, wherein determining the user instruction based on the sensor data comprises determining a pattern in the sensor data and determining that the pattern matches a pattern associated with the user instruction.

40. The ophthalmic system of claim 30, wherein determining the user instruction based on the sensor comprises determining a signature in the sensor data and determining that the signature matches a signature associated with the user instruction.

41. The ophthalmic device of claim 30, further comprising an amplifier operatively associated with the sensor.

42. The ophthalmic device of claim 30, further comprising an analog-to-digital converter operatively associated with the sensor.

43. The ophthalmic device of claim 30, wherein the sensor comprises a displacement sensor.

44. The ophthalmic device of claim 30, wherein determining the user instruction based on the sensor data comprises filtering out sensor data indicative of user movements not intended as a user instruction.

45. A method comprising:
receiving, by an ophthalmic device disposed in or on an eye of a user from a sensor configured to be disposed at least partially in a mouth of the user, sensor data;
determining a user instruction based on the sensor data; and
causing the ophthalmic device to be controlled based on the user instruction.

46. The method of claim 45, wherein the sensor is disposed in or on a cap configured to attach to a tooth of the user.

47. The method of claim 45, wherein the sensor comprises a contact sensor.

48. The method of claim 45, wherein the sensor data is indicative of a contact with one or more of a tooth or a tongue of a user.

49. The method of claim 45, wherein the ophthalmic device comprises an ophthalmic lens and a variable-optic element configured to change a refractive power of the ophthalmic lens, and wherein causing the ophthalmic device to be controlled based on the user instruction comprises causing the variable-optic element to be controlled based on the user instruction.

50. The method of claim 45, wherein the ophthalmic device comprises a transceiver configured to transmit data from the ophthalmic device, and wherein causing the ophthalmic device to be controlled based on the user instruction comprises causing the transceiver to be controlled based on the user instruction.

51. The method of claim 45, wherein the sensor data is indicative of a vibration.

52. The method of claim 51, wherein the vibration comprises one or more of a sound made by the user or speech by the user.

53. The method of claim 45, wherein the sensor data is indicative of one or more of chattering, moving, grinding, or clenching a tooth of the user.

54. The method of claim 45, wherein determining the user instruction based on the sensor data comprises determining a pattern in the sensor data and determining that the pattern matches a pattern associated with the user instruction.

55. The method of claim 45, wherein determining the user instruction based on the sensor comprises determining a signature in the sensor data and determining that the signature matches a signature associated with the user instruction.

56. The method of claim 45, wherein the ophthalmic device comprises an amplifier operatively associated with the sensor.

57. The method of claim 45, wherein the ophthalmic device comprises an analog-to-digital converter operatively associated with the sensor.

58. The method of claim 45, wherein the sensor comprises a displacement sensor.

59. The method of claim 45, wherein determining the user instruction based on the sensor data comprises filtering out sensor data indicative of user movements not intended as a user instruction.

60. The method of claim 59, wherein filtering out sensor data indicative of user movements not intended as a user instruction comprises filtering out sensor data in one or more of a time domain or a frequency domain.
